(19) United States
(12) Patent Application Publication
(10) Pub. No.: US 2015/0017655 A1
(43) Pub. Date: Jan. 15, 2015

(54) LABEL-FREE SEQUENCING METHOD FOR SINGLE NUCLEIC ACID MOLECULE

(71) Applicant: NATIONAL CHIAO TUNG UNIVERSITY, HSINCHU CITY (TW)

(72) Inventors: GUE-WHA HUANG, MIAOLI COUNTY (TW); MENG-YEN HUNG, MIAOLI COUNTY (TW); YU-SHIUN CHEN, YILAN COUNTY (TW)

(73) Assignee: NATIONAL CHIAO TUNG UNIVERSITY, HSINCHU CITY (TW)

(21) Appl. No.: 14/264,523

(22) Filed: Apr. 29, 2014

(30) Foreign Application Priority Data
Jul. 9, 2013 (TW) 102124556

(51) Int. Cl. C12Q 1/68

(52) U.S. Cl. CPC C12Q 1/6869
USPC 435/6.19

(57) ABSTRACT

A label-free sequencing method for a single molecular nucleic acid is provided. The primer is paired with the nucleic acid template to be assembled to a polymerase. When the nucleotides are added, the electrical conductance signal is measured by the polymerase being connected to the protein transistor to determine the sequences of the nucleic acid template. The trajectory of the measured electrical conductance signal contains plateaux with obvious spikes, which is used to identify four types of the nucleotides and their bases. Furthermore, the sequencing method is suitable for sequencing of complex nucleic acids.

This application claims priority for Taiwan patent application no. 102124556 filed at Jul. 9, 2013, the content of which is incorporated by reference in its entirely.

### BACKGROUND OF THE INVENTION

1. Field of the Invention

The present invention relates to a sequencing technology for a single molecule, particularly to a sequencing method measuring conductance to sequence a single unlabelled nucleic acid molecule.

2. Description of the Related Art

The emergence of personalized medicine indicates evolution from traditional medicine to personal genetic information-dependent medicine. The key of personalized medicine is a technology able to fast sequence DNA in high throughput and low cost. In the past decade, a new-generation sequencing technology has been developed, based on the arrayed reactions that sequence amplified DNA targets. Compared with the former-generation (Sanger) sequencing method, the new-generation sequencing technology can obviously decrease the time required to completely sequence a human genome. However, the short read length and high error rate limits the application of the new technology to sequencing unknown genomes.

One of the third-generation sequencing technologies is the single-molecule sequencing, which does not require amplification, ligation or cloning and is expected to provide single-molecule resolution, long read length and negligible error rate, together with a reduction in cost. Such methods typically involve cyclic reactions using fluorescent substrates that are monitored by optical imaging, and have, for example, been used to sequence the M13 viral genome.

An alternative third-generation technology is the nanopore sequencing, which uses a special protein to perforate nanopores in a membrane, and which identifies the sequence of nucleotides (T, C, G, and A) of a DNA molecule by measuring the modulations in the ionic current across a synthetic or biological pore while the DNA molecule is driven through it under an applied potential. This approach has been used to read DNA at single-nucleotide resolution by using Φ29 DNA polymerase (F29) to control the rate of DNA translocation through an MspA nanopore. Oxford Nanopore Technologies has also reportedly used a prototype nanopore device to decode a viral genome in a single pass of a complete DNA strand.

The commercialized third-generation technology is the only method currently comparable to the next-generation sequencing methods. However, the short read length and high error rate thereof have yet to be solved.

### SUMMARY OF THE INVENTION

The primary objective of the present invention is to provide a label-free sequencing method for a single nucleic acid molecule, which incorporates unlabelled nucleotides into a nucleic acid template, assembles the template to a polymerase, and measures the conductance of the polymerase to sequence the nucleic acid molecule. The method of the present invention is not only adaptive to different polymerases but also able to decode various difficult nucleic acid sequences with very high accuracy.

To achieve the abovementioned objective, the present invention proposes a label-free sequencing method for a single nucleic acid molecule, which comprises steps: providine, a protein transistor including two electrodes and at least two gold nanoparticles respectively connected with the two electrodes, wherein a bias is applied to the two electrodes to make a first antibody self-assemble to the two gold nanoparticles; connecting a polymerase with the first antibody; introducing a nucleic acid template, pairing a primer with the nucleic acid template, and assembling the template to a polymerase; adding one or more types of unlabelled nucleotides to react with the polymerase to synthesize a complementary nucleic acid; using the protein transistor to synchronically detect the conductance signals generated by the reaction of the polymerase and obtain a conductance trajectory; and determining the sequence of the nucleic acid template according to the conductance trajectory.

In the present invention, the conductance signals of the reaction of the polymerase is detected via connecting the polymerase with the protein transistor, wherein the polymerase is connected with a second antibody molecule firstly; next the second antibody is connected with the first antibody molecule; next the first antibody is connected with the two gold nanoparticles and thus electrically connected with the source electrode and drain electrode of the protein transistor. After addition of the nucleotides, the conductance trajectory presented by the polymerase has reaction plateaux with heights of 3-6 pA, which can be recognized very easily. Each plateau is exactly corresponding to a nucleotide being read. The present invention can read about 22 nucleotides per second. The spikes of the reaction plateaux have obvious features sufficient to discriminate four different types of nucleotides. Further, the present invention can use different polymerases to decode difficult sequences, such as homopolymers. Furthermore, the present invention can arrange several self-assembling protein transistors in an identical chip to simultaneously sequence several nucleic acid templates.

Below, the embodiments are described in detail to make easily understood the objectives, technical contents, characteristics and accomplishments of the present invention.

### BRIEF DESCRIPTION OF THE DRAWINGS

FIG. 1 shows a flowchart of a label-free sequencing method for a single nucleic acid molecule according to one embodiment of the present invention;

FIG. 2 schematically shows a protein transistor used to realize a label-free sequencing method for a single nucleic acid molecule according to one embodiment of the present invention;

FIG. 3a shows a conductance trajectory obtained in a reaction catalyzed by Φ29 DNA polymerase connected with a protein transistor according to one embodiment of the present invention;

FIG. 3a-1, FIG. 3a-2 and FIG. 3a-3 are respectively locally-enlarged views of Inset 1, Inset 2 and Inset 3 of FIG. 3a.

FIG. 3b and FIG. 3c are respectively conductance trajectories generated in using Φ29 DNA polymerase to read a nucleic acid template carrying GATC repeats and a nucleic acid template carrying TTCCGGAA repeats according to one embodiment of the present invention, wherein the nucleotide type is designated below each reaction plateau;

FIGS. 4a-4d respectively elementary patterns of reaction plateaux of nucleotides G, T, C and A according to one embodiment of the present invention;

FIGS. 5a-5d respectively show conductance trajectories obtained via using Φ29 DNA polymerase (F29), T4 DNA polymerase (T4), T7 DNA polymerase (T7) and DNA polymerase I (Pol I) of colon bacillus to sequence a nucleic acid template of Oligo 3 according to one embodiment of the present invention; and

FIG. 6 shows a conductance trajectory obtained via using Φ29 DNA polymerase to sequence a nucleic acid carrying a homopolymer according to one embodiment of the present invention.

### DETAILED DESCRIPTION OF THE INVENTION

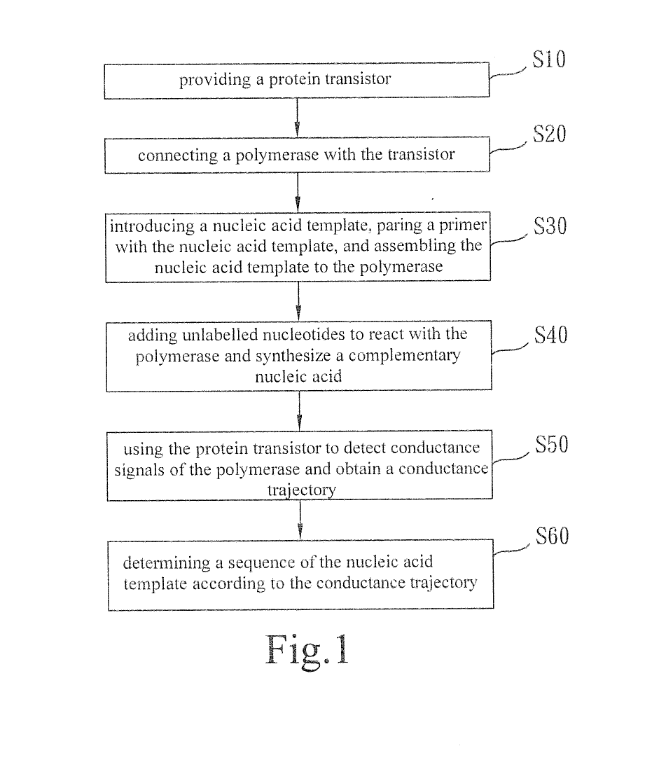

The present invention provides a label-free sequencing method for a single nucleic acid molecule, which uses a polymerase to pair unlabelled nucleotides with a nucleic acid template and uses conductance signals to sequence a single nucleic acid molecule, such as a single molecule of DNA or RNA. Refer to FIG. 1 for a flowchart of a label-free sequencing method for a single nucleic acid molecule according to one embodiment of the present invention. The method of the present invention comprises Step S10-S60.

In Step S10, Prepare a protein transistor, which provides stable conductance readings and is designed to hold a polymerase during synthesis of a new strand.

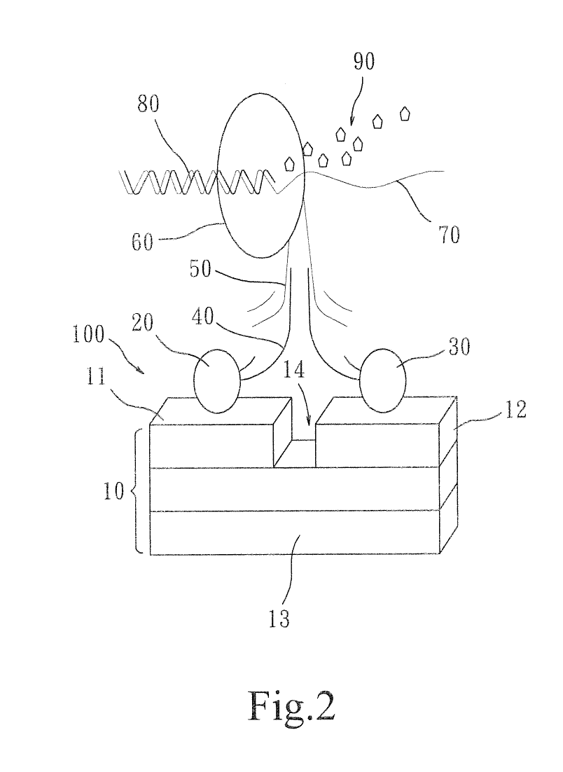

Refer to FIG. 2 for an embodiment of a protein transistor. The protein transistor 100 includes a transistor 10 and at least two gold nanoparticles 20 and 30. The transistor 10 has a source electrode 11, a drain electrode 12 and a gate electrode 13. An electron beam photolithographic technology is used to fabricate a nanochannel 14 with a width of about 10 nm. The two gold nanoparticles 20 and 30 (each has a diameter of 5 nm) are brought to the edges of the source electrode 11 and the drain electrode 12 by AFM (Atomic Force Microscope). PDMS (polydimethylsiloxane) is used to cover the gold nanoparticles 20 and 30, preventing the elements from being damaged and generating a preformed liquid channel (having a width of 100 nm and a depth of 20 nm). FIG. 2 shows a first antibody molecule 40, which may be an immunoglobulin. The first antibody molecule 40 (the immunoglobulin) is transported through the liquid channel at a flow rate of 0.1 μl/sec, wherein the liquid contains the immunoglobulin at a concentration of 1 pg/ml. A bias is applied to the source electrode H and the drain electrode 12 through the gate electrode 13 to make the first antibody molecule 40 pass through the nanochannel 14 and self-assemble to the two gold nanoparticles 20 and 30.

In Step S20, connect a polymerase 60 with a second antibody molecule 50, and then connect the second body molecule 50 with the first antibody molecule 40. Alternatively, directly connect the polymerase 60 with the first antibody molecule 40. The second antibody molecule 50 may also be an immunoglobulin. The polymerase 60, such as a DNA polymerase, is an enzyme to catalyze the synthesis of DNA. The polymerase 60 is Φ29 DNA polymerase, T4 DNA polymerase, T7 DNA polymerase, or DNA polymerase I.

The Φ29 DNA polymerase is a replicative polymerase with long processivity and low error rate. The Φ29 DNA polymerase is chemically cross-linked to the second antibody molecule 50; next the second antibody molecule 50 is connected with the Fc domain of the first antibody molecule 40 on the protein transistor 100; then the first antibody molecule 40 is bonded to the gold nanoparticles 20 and 30 respectively on the source electrode 11 and the drain electrode 12. The self-assembly process can be monitored with measuring conductance and will be described in detail thereinafter.

In Step S30, introduce a nucleic acid template 70, pair a primer 80 with the nucleic acid template 70, and assemble them to the polymerase 60. In the present invention, the nucleic acid template 70 is a single-strand DND (ssDNA), a double-strand DNA (dsDNA) or an RNA.

In Step S40, add one or more types of unlabelled nucleotides 90 to react with the polymerase 60 and generate a complementary nucleic acid. In the present invention, the unlabelled nucleotides 90 are dNTPs, including dTTP, dATP, dCTP, and dGTP. During the reaction, a nucleotide 90 (dNTP) complementary to the nucleic acid template 70 is chosen according the base-pairing principle to form a phosphodiester bonded to the 3′-OH of the primer 80 and release pyrophosphate. Before dissociating from the nucleic acid template 70, the chain elongates as the DNA polymerase 60 proceeds along the nucleic acid template 70. The interaction between the nucleotides 90 (dNTP) and the DNA polymerase 60 exhibits a classical Michaelis-Menten mechanism consisting of steps of substrate-binding (base-pairing) and bond-formation.

In Step S50, while the nucleotides 90 participate in the synthesis reaction described in Step S40, detect the conductance signals between the source electrode 11 and the drain electrode 12 to learn the conductance variation of the polymerase 60 and obtain the conductance trajectory of the polymerase 60.

In Step S60, determine the sequence of the nucleic acid template 70 according to the conductance trajectory.

Below will be described in detail the experiments of conductance detection and label-free sequencing of a single nucleic acid molecule according to the conductance trajectory.

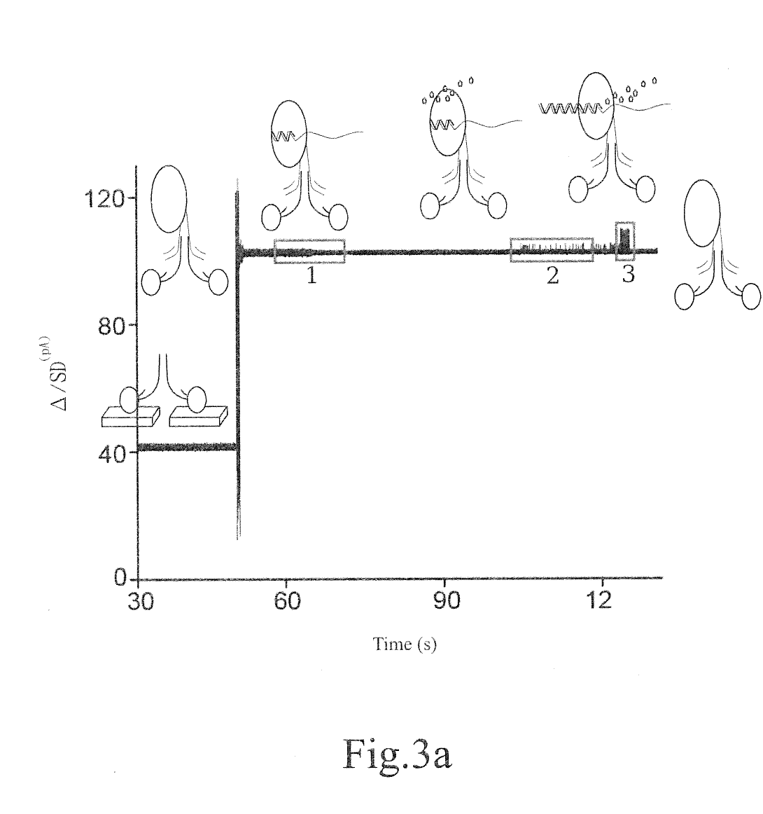

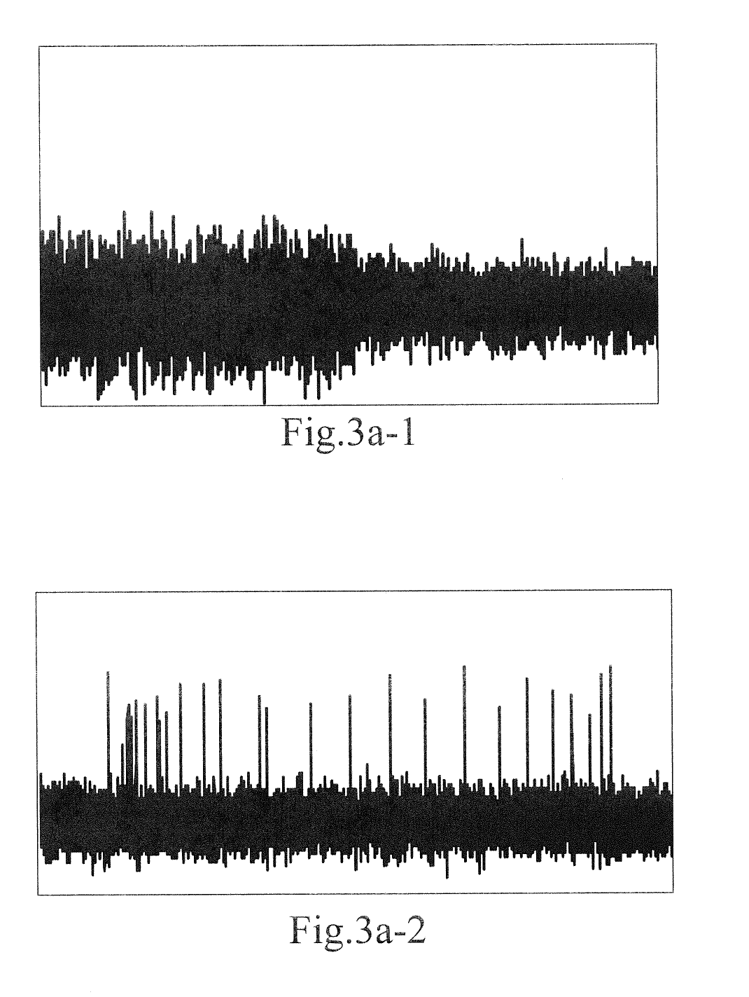

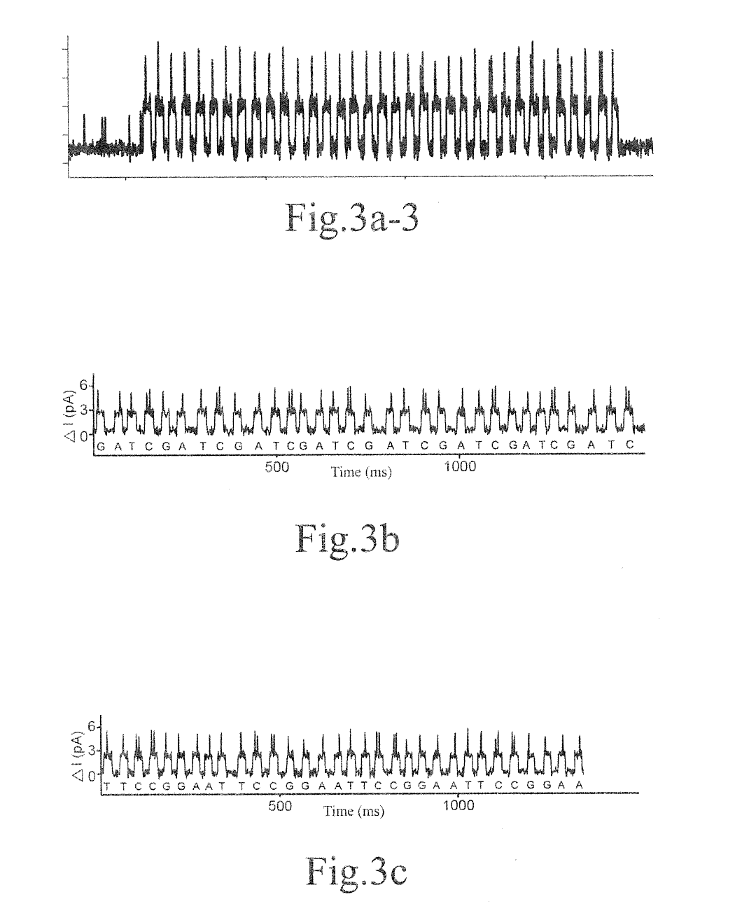

The present invention uses a protein transistor to monitor the conductance variation of a polymerase and recognize different nucleotides. Refer to FIG. 3a for a conductance trajectory, which shows the process that Φ29 DNA polymerase is connected with a protein transistor and undertakes reactions and the conductance signals corresponding to the reactions. In the present invention, while a bias is applied to the gate electrode and a stable source-drain current (ISD) is detected, it indicates that the first antibody molecule (immunoglobulin) can successfully pass through the nanochannel and self-assemble to the two gold nanoparticles. FIG. 3a shows that the initial conductance signal of the protein transistor is about 43 pA.

Next, a conjugate of the Φ29 DNA polymerase, which is purified by column chromatography, is carried to the protein transistor and attached to the Fc terminal of the first antibody molecule on the protein transistor. While the source-drain voltage (VSD) is 9.0 V and the gate voltage (VG) is 3.0 V, the attachment of the Φ29 DNA polymerase conjugate induces an irreversible current rise by about 60 pA. Meanwhile, a prominent conductance signal appears in the conductance trajectory. The conductance signal will finally settle at a stable value of 102 pA with a noise level of about 5 pA.

In order to obtain a pico-ampere signal, all measurements are performed in a shielding room to minimize electromagnetic and radiofrequency interference. In order to reduce signal decay, superconducting materials are used for the interface between the transistor and probes of signal-output terminals. The dynamic response of the conductance signals is measured by sending a high-frequency laser pulse to the quantum dots of protein transistor and measuring the photon-induced fluctuation in the conductance signal. The laser waveform at a frequency of 1.7×10⁹s⁻¹ can be detected by means of electrical conductance with fidelity. It indicates that the system of the present invention can provide a sub-nanosecond dynamic response. The turnover rate of Φ29 DNA polymerase ranges from 20 to 150 nucleotides (nt) per second, and the sequencing reaction occurs within a millisecond time scale. Thus, the time bin is set to be 1 nanosecond during the measurement.

The sequential incorporation of nucleotides, as well as the identities of the four different nucleotides, can be detected by their characteristic conductance responses. A synthetic template carrying GATC repeats is annealed with a complementary primer and loaded onto the immobilized Φ29 DNA polymerase. If sufficient time is allowed, the fluctuation of noise will eventually stabilize with the noise level decreasing to 1 pA (shown in FIG. 3a, Inset 1). Synthesis of the complementary DNA strand is triggered by passing 1 μM dNTPs through the sequencing platform. The conductance variation during polymerization is the key to determine the sequence of nucleic acids. The conductance trajectory is then recorded during polymerization. Spikes (shown in FIG. 3a, Inset 2) with a height of 1.5-3 pA appears stochastically at the start of the conductance trajectory after the injection of dNTPs, revealing the reversible binding of the substrate dNTPs and the polymerase. These spikes are probably caused by the rapid binding and disassociation of the polymerase and nucleotides. The disordered spikes are followed by grouped but well-separated plateaux that are 3-6 pA in height (shown in FIG. 3a, Inset 3). The shape of the plateaux can be used to identify the stages of sustained enzyme-substrate binding, catalyzed reactions and pyrophosphate release. The appearance of sequential plateaux indicates stepwise base pairing and nucleotide incorporation into the growing strand. The rate of plateau formation is about 22 nt which matches the turnover rate of Φ29 DNA polymerase at a temperature of 25° C. The DNA replication continues to till in the complementary sequences until falloff of the nucleic acid template. After the polymerase completes synthesis of the double-strand DNA, the conductance trajectory falls to the original inactive level.

The binding of a nucleotide to the active site of the (Φ29 DNA polymerase promotes conductance. The binding between the nucleotide and the polymerase is followed by bond formation, release of pyrophosphate, sliding down of the double-stranded DNA, active site evacuation (creating room for the next nucleotide, and binding of the nucleotide and the polymerase. One complete reaction cycle appears as a plateau in the conductance trajectory. The four different nucleotides are distinguished by their characteristic spike patterns. Refer to FIG. 3b and FIG. 3c. Herein, the nucleic acid template carrying repeated GATC and the nucleic acid template carrying repeated TTCCGGAA are used to demonstrate the characteristics of the spikes. G nucleotide, T nucleotide and A nucleotide exhibit a single spike the plateau, while C nucleotide exhibits multiple spikes. These spikes have a height of about 5-6 pA. With a sudden increase or decrease in conductance, spikes indicate a temporary change in electrostatic organization.

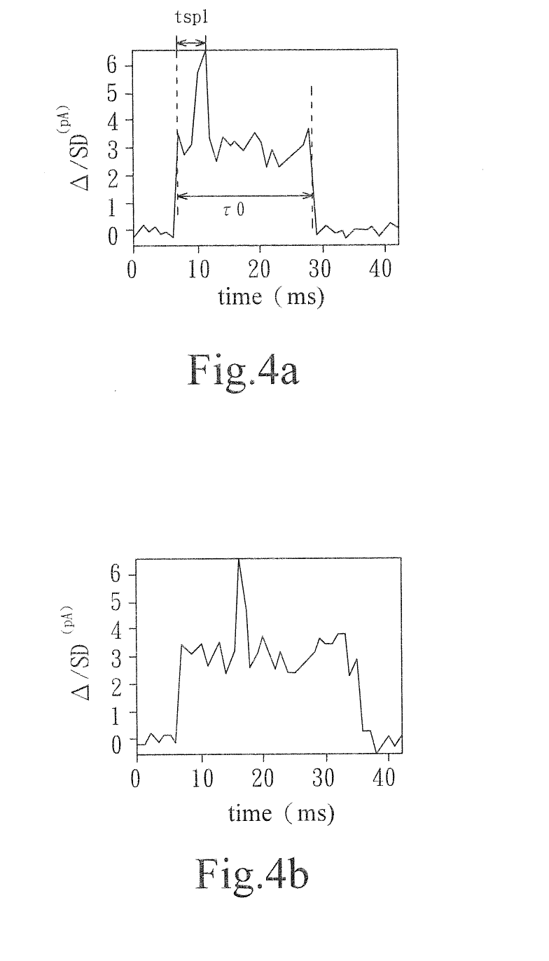

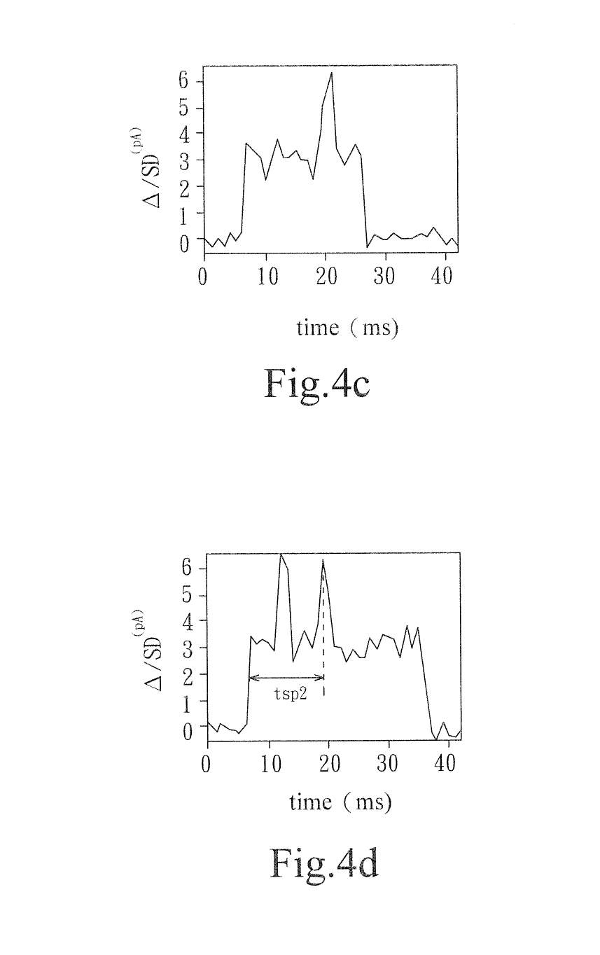

The present invention performs statistics of more than fifty thousands of the patterns of reaction plateaux and obtains the following results. FIGS. 4a-4d are randomly selected from the primitive data to respectively exemplify the reaction plateaux of G nucleotide, T nucleotide, A nucleotide and C nucleotide. A typical pattern of a reaction plateau includes the time from the start point of the reaction plateau to the peak of the first spike or the second spike (tsp1 or tsp2). From the results, it is learned: the time from the start point of the reaction plateau to the peak of the spike (tsp1) of G nucleotide is 3.1±0.13 ms; tsp1 of T nucleotide is 9.3±0.11 ms; tsp1 of A nucleotide is 13.1±0.14 ms, tsp1 of C nucleotide is 5.2±0.15 ms, and tsp2 of C nucleotide is 12.2±0.12 ms. The spike patterns seem neither relevant to the number of hydrogen bonds nor relevant to the chemical composition of the nucleosides. The widths of the plateaux can be used to distinguish pyrimidines (T and C) and purines (G and A). The plateau width of pyrimidines (T and C) is longer than that of purines (G and A). The widths (τ0) of the plateaux are 22.3±2.4, 29.5±2.2, 20.3±2.1 and 30.2±2.3 ms for G nucleotide, T nucleotide, A nucleotide and C nucleotide respectively. The focused width distribution of the reaction plateaux indicates that the catalytic activity of the polymerase is constant and non-stochastic.

Base-calling is verified by giving one type of nucleotide at a time. The characteristic electrical signature appears only when the corresponding nucleotide is provided. The reaction plateaux would not appear unless the correct substrate reacts with the activated site of the polymerase. For example, in synthesis of G nucleotide, injection of dGTP causes appearance of a reaction plateau. While the synthesis reaction of G nucleotide is terminated and the polymerase is displaced, dGTP is nor more the correct substrate. In such a case, addition of dGTP would not cause appearance of a reaction plateau. Furthermore, if the polymerization is terminated by dideoxynucleotide, addition of dNTP gives only binding spikes, without any reaction plateaux. The nucleotides dGTP, dATP and dTTP are used in the sequencing experiment, bonded to the frequently-seen primers according to randomly mixed nucleic acid templates, and assembled to the protein transistor by Φ29 DNA polymerase. The results show that sequencing randomly mixed templates also exhibit the accuracy of base-calling. From shape analysis, it is found that the patterns starting from the beginning of the plateaux and extending over 90% of the plateaux are consistent. Variations in width and shape frequently occur at the end of the plateaux, which is after the last spike in the cases of A nucleotide and C nucleotide. This result demonstrates that single-molecule sequencing can be achieved by monitoring the conductance of a polymerase during the synthesis of a growing DNA strand.

The present invention also makes a research to further study the association between the nucleotides and the corresponding plateau shapes via examining the conductance trajectories of the other DNA polymerases.

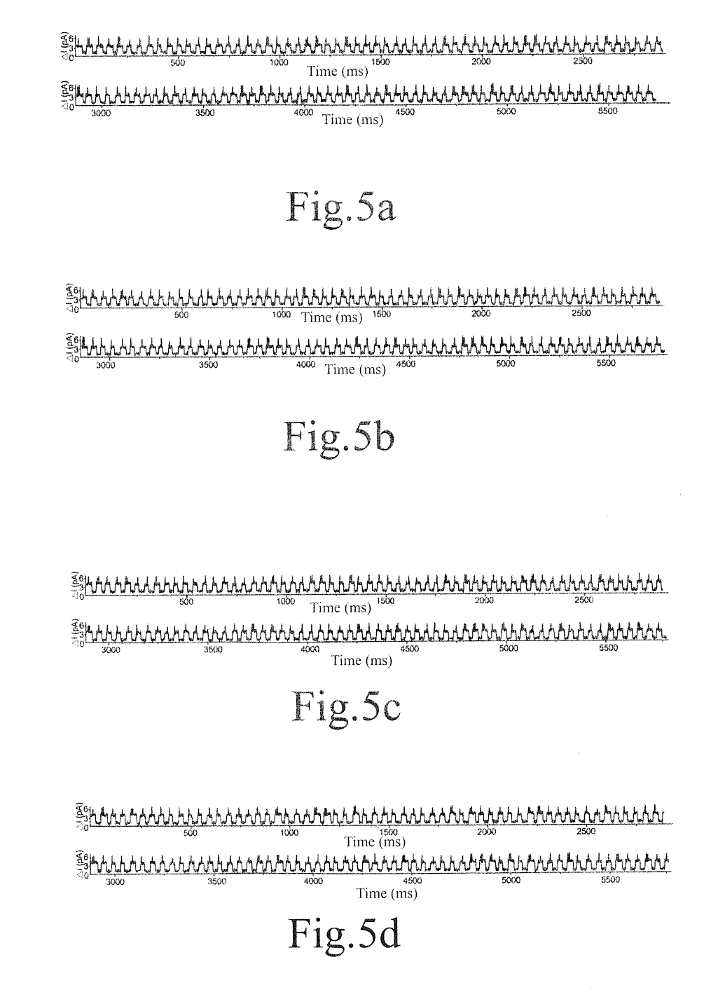

Refer to FIGS. 5a-5d. These conductance trajectories are obtained via using Φ29 DNA polymerase (F29), T4 DNA polymerase (T4), T7 DNA polymerase (T7) and DNA polymerase I (Pol I) of colon bacillus to sequence a nucleic acid template of Oligo 3, whose sequence is expressed by
5′-aagaagttacgattcgcgggtcctcagaatgaacattcagagaatcatactaacaccagaaacca gtacataggccacagcgttcttcaacgccggtacgaattactccccattgaaga
cgccgcggagccaag-3′. Refer to Table. 1. The plateau widths and heights obtained from F29, T4, T7 and Pol I share a high degree of similarity, with only minor variations. The shape of the plateaux corresponding to nucleotides G, T, A and C remains distinguishable. The association between plateaux and nucleotides for various polymerases indicates that the molecular mechanisms of base pairing and bond formation are common features shared by DNA polymerases. Under conditions where the plateau shapes are sufficient to sequence a DNA molecule, the inherently long processivity and low error rate of the F29, T4 and T7 polymerases make them good candidates for genomic sequencing.

TABLE 1

| | Plateau Height Hr (pA) | | | | Plateau Width τ0 (ms) | | | |
|---|---|---|---|---|---|---|---|---|
| | G | T | A | C | G | T | A | C |
| Φ29 | 3.1 ± 0.41 | 3.01 ± 0.41 | 3.0 ± 0.43 | 3.1 ± 0.42 | 22.3 ± 2.4 | 29.5 ± 2.2 | 20.3 ± 2.1 | 30.2 ± 2.3 |
| T4 | 3.2 ± 0.42 | 3.2 ± 0.58 | 3.1 ± 0.43 | 3.3 ± 0.54 | 22.2 ± 2.5 | 29.2 ± 2.4 | 20.1 ± 2.3 | 30.5 ± 2.2 |
| T7 | 3.3 ± 0.42 | 3.6 ± 0.43 | 3.1 ± 0.4 | 3.7 ± 0.41 | 23.1 ± 2.3 | 26.4 ± 2.3 | 19.2 ± 2.1 | 28.3 ± 2.2 |
| Pol I | 3.4 ± 0.8 | 3.7 ± 0.68 | 3.2 ± 0.72 | 3.8 ± 0.67 | 20.1 ± 2.4 | 25.4 ± 2.4 | 26.2 ± 2.3 | 33.2 ± 2.6 |

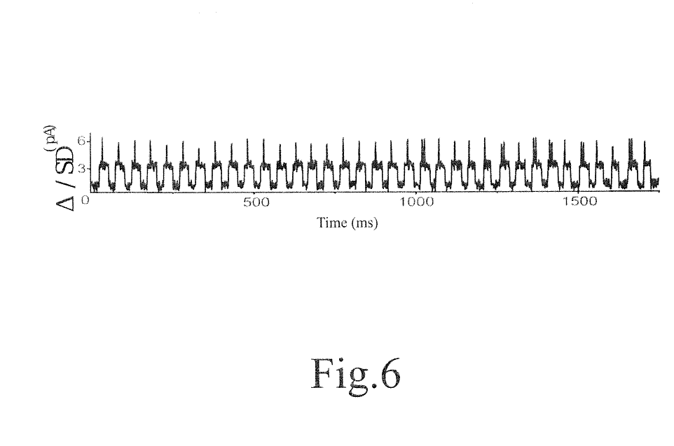

Nucleic acid templates containing a stretch of a single nucleotide are known to be difficult for sequencing, and such templates frequently give read errors in many sequencing technologies. Refer to FIG. 6. To explore the utility of the new sequencing platform of the present invention, Φ29 DNA polymerase is used to sequence a template containing 20 consecutive T nucleotides. The nucleic acid template carrying the homopolymer is sequenced with F29. The decoded sequence (5′ to 3′) is (t) 20 caggetccgcggcg. The results indicate that the protein transistor platform is indeed capable of resolving 20 T nucleotides without ambiguous reading.

The conductance trajectories measured by the present invention are consistent with previous studies examining the kinetics of DNA polymerases and single-molecular enzymes. The Michaelis-Menten mechanism proposes a reversible binding step that occurs before the formation of an enzyme-substrate complex, which is followed by catalysis and product release. This mechanism is corroborated by the observed binding spikes and groups of reaction plateaux (shown in FIG. 3a, Insets 2 and 3). Spikes are a result of rapid increases and decreases in conductance corresponding to reversible substrate binding. Once a stable enzyme-substrate complex is formed, catalytic events continue to occur and can be observed by examining the reaction plateaux. The onset of each reaction is stochastic, and this is consistent with observations made using single-molecule fluorescence. However, the sharp distribution of plateau widths indicates that catalysis obeys precisely designed molecular steps.

The experimental details of the present invention are provided below.

(1) Materials and Methods

Φ29 DNA polymerase, T4 DNA polymerase, T7 DNA polymerase, and DNA polymerase I (E. coli) are purchased from NEB or Invitrogen. The standard reaction buffers for the Φ29 DNA polymerase (50 mM Tris-HCl pH 7.5, 10 mM MgCl2, 10 mM (NH4)2SO4, 4 mM DTT), T4 DNA polymerase (33 mM Tris-acetate pH 7.9, 66 mM sodium acetate, 10 mM magnesium acetate, 1 mM DTT), 17 DNA polymerase (20 mM Tris-HCl pH 7.5, 10 mM MgCl2, 1 mM DTT), and DNA Polymerase I (10 mM Tris-HCl pH 7.9, 50 mM NaCl, 10 mM MgCl2, 1 mM DTT) are made according to the supplier's specifications. The reaction buffer used in the experiment is made via diluting the standard buffer 1,000,000 folds. Ensemble experiments are performed to verify if the diluted reaction buffer and diluted magnesium affects polymerase activity. The polymerase activity remains unchanged in the presence of diluted reaction buffer. The activity of Φ29 DNA polymerase is measured via comparing fluorescence in a buffer solution diluted one fold. The sequencing results of a single molecule in the buffer solutions respectively diluted 10², 10³, 10⁴, 10⁵ or 10⁶ folds prove that the reaction rate of the polymerase is the same before and after dilution. In the present invention, the experiments of the label-free sequencing method for single nucleic acid molecule are conducted in the diluted buffer.

(2) Conjugation of Polymerases

A rabbit anti-mouse IgG (H+L) antibody (ZyMax™ Grade, Invitrogen, CA) is reconstituted in 10 mM phosphate buffered saline (pH 7.4) to a final concentration of 2 mg/ml. 5% glutaraldehyde (Sigma) is added to the antibody solution at a final concentration of 0.2%. Conjugation is performed by mixing 0.5 mg activated antibody with 1.5 mg DNA polymerases and 100 μl phosphate buffer followed by incubation at 25° C. for 2 hours. The reaction is terminated by adding the phosphate buffer to a final volume of 1 ml. The conjugates are purified by passage through a protein A column. The supernatants are further purified by high pressure liquid chromatography (HPLC) (Discovery BIO GFC 100 HPLC Column L×I.D. 5 cm×4.6 mm; Discovery R BIO GFC 100 L×I.D. 30 cm×4.6 mm).

In conclusion, the present invention proposes a label-free sequencing method for a single nucleic acid molecule, which determines the sequence of a single nucleic acid molecule according to the conductance signals occurring while the polymerase is assembled to the protein transistor. While nucleotides participate in synthesis, the conductance signals generated by assemblage of the polymerase to the protein transistor are used to determine the sequence of the nucleic acid template. The trajectory of the conductance signals includes a plurality of reaction plateaux each containing at least one characteristic spike. The reaction plateaux containing the characteristic spikes are respectively corresponding to four nucleotides and the bases thereof. The present invention is adaptive to different polymerases and able to decode various difficult nucleic acids, including a nucleic acid containing 20 consecutive T nucleotides. Experiments prove that the present invention can read more than 50000 nucleotides without even one error. It indicates that the present invention has remarkable precision. Further, the present invention can use a chip containing a plurality of self-assembling protein transistors to sequence a plurality of nucleic acid templates simultaneously.

The embodiments described above are only to exemplify the present invention but not to limit the scope of the present invention. Any equivalent modification or variation according to the spirit of the present invention is to be also included within the scope of the present invention.

What is claimed is:

1. A label-free sequencing method for a single nucleic acid molecule, comprising
Step (a): providing a protein transistor including two electrodes and at least two gold nanoparticles connected with said two electrodes, wherein a bias is applied to said two electrodes to make a first antibody molecule self-assemble to said two gold nanoparticles;
Step (b): connecting a polymerase with said first antibody molecule;
Step (c): introducing a nucleic acid template, paring a primer with said nucleic acid template, and assembling said nucleic acid template to said polymerase;
Step (d): adding one or more unlabelled nucleotides to react with said polymerase and synthesize a complementary nucleic acid;
Step (e): using said protein transistor to detect a plurality of conductance signals of said polymerase and obtain a conductance trajectory; and
Step (f): determining a sequence of said nucleic acid template according to said conductance trajectory.

2. The label-free sequencing method for a single nucleic acid molecule according to claim 1, wherein said protein transistor further comprises a gate electrode, and said two electrodes are respectively a drain electrode and a source electrode, and wherein a nanochannel is fabricated between said drain electrode and said source electrode, and wherein a bias is applied to said drain electrode and said source electrode through said gate electrode to make said first antibody molecule pass through said nanochannel and self-assemble to said two gold nanoparticles.

3. The label-free sequencing method for a single nucleic acid molecule according to claim 1, wherein said first antibody molecule is an immunoglobulin molecule.

4. The label-free sequencing method for a single nucleic acid molecule according to claim 1, wherein said polymerase is connected with a second antibody molecule, and said second antibody molecule is connected with said first antibody molecule.

5. The label-free sequencing method for a single nucleic acid molecule according to claim 4, wherein said second antibody molecule is an immunoglobulin molecule.

6. The label-free sequencing method for a single nucleic acid molecule according to claim 1, wherein said one or more unlabelled nucleotides are one or more deoxynucleoside-triphosphates (dNTP).

7. The label-free sequencing method for a single nucleic acid molecule according to claim 6, wherein said one or more unlabelled nucleotides are selected from a group consisting of deoxythymidine-triphosphate (dTTP), deoxyadenosine-triphosphate (dATP), deoxycytidine triphosphate (dCTP) and deoxyguanosine-triphosphate (dGTP).

8. The label-free sequencing method for a single nucleic acid molecule according to claim 1, wherein said nucleic acid template is a single-strand DNA (ssDNA), a double-strand DNA (dsDNA), or an RNA.

9. The label-free sequencing method for a single nucleic acid molecule according to claim 1, wherein said polymerase is selected from a group consisting of Φ29 DNA polymerase, T4 DNA polymerase, T7 DNA polymerase and DNA polymerase I.

10. The label-free sequencing method for a single nucleic acid molecule according to claim 1, wherein in said Step (d), a high-frequency laser pulse is applied to said polymerase to measure photon-induced fluctuation in said conductance signals.

11. The label-free sequencing method for a single nucleic acid molecule according to claim 1, wherein in said conductance trajectory, a plurality of spikes appears stochastically at start, and a plurality of well-separated reaction plateaux about 3-6 pA in height appears then, and wherein shapes of said reaction plateaux are used to identify one or more types of nucleotides, a sequence of said nucleotides, and one or more bases corresponding to said nucleotides.

12. The label-free sequencing method for a single nucleic acid molecule according to claim 11, wherein each of said reaction plateaux of said nucleotides has one or more spikes having a height of 5-6 pA, and wherein said reaction plateau of each of G nucleotide, T nucleotide and A nucleotide has a single spike, and said reaction plateau of C nucleotide has multiple spikes.

13. The label-free sequencing method for a single nucleic acid molecule according to claim 12, wherein said conductance trajectory generated in a sequencing process using said Φ29 DNA polymerase has characteristic time intervals from a rising point of said reaction plateau to a peak of said spike or peaks of said spikes, and wherein said characteristic time interval of G nucleotide is 3.1±0.13 ms; said characteristic time interval of T nucleotide is 9.3±0.11 ms; said characteristic time interval of A nucleotide is 13.1±0.14 ms; a first said characteristic time interval of C nucleotide is 5.2±0.15 ms, and a second said characteristic time interval of C nucleotide is 12.2±0.12 ms.

14. The label-free sequencing method for a single nucleic acid molecule according to claim 12, wherein said conductance trajectory generated in a sequencing process using said Φ29 DNA polymerase has characteristic plateau widths, which are respectively 22.3±2.4 ms, 29.5±2.2 ms, 20.3±2.1 ms and 30.2±2.3 ms for G nucleotide, T nucleotide, A nucleotide and C nucleotide.

15. The label-free sequencing method for a single nucleic acid molecule according to claim 12, wherein said conductance trajectory generated in a sequencing process using said T4 DNA polymerase has characteristic plateau widths, which are respectively 22.2±2.5 ms, 29.2±2.4 ms, 20.1±2.3 ms and 30.5±2.2 ms for G nucleotide, T nucleotide, A nucleotide and C nucleotide.

16. The label-free sequencing method for a single nucleic acid molecule according to claim 12, wherein said conductance trajectory generated in a sequencing process using said T7 DNA polymerase has characteristic plateau widths, which are respectively 23.1±2.3 ms, 26.4±2.3 ms, 19.2±2.1 ms and 28.3±2.2 ms for G nucleotide, T nucleotide, A nucleotide and C nucleotide.

17. The label-free sequencing method for a single nucleic acid molecule according to claim 12, wherein said conductance trajectory generated in a sequencing process using said DNA polymerase I (Pol I) has characteristic plateau widths, which are respectively 20.1±2.4 ms, 25.4±2.4 ms, 26.2±2.3 ms and 33.2±2.6 ms for G nucleotide, T nucleotide, A nucleotide and C nucleotide.

18. The label-free sequencing method for a single nucleic acid molecule according to claim 12, wherein said characteristic plateau widths of T nucleotide and C nucleotide are larger than said characteristic plateau widths of G nucleotide and A nucleotide.

19. The label-free sequencing method for a single nucleic acid molecule according to claim 12, wherein variation in width and shape occurs after appearance of the last spike for A nucleotide and C nucleotide.

20. The label-free sequencing method for a single nucleic acid molecule according to claim 1, wherein a plurality of said protein transistors is simultaneously arranged in an identical chip to respectively sequence a plurality of nucleic acid templates.
